(19) United States
(12) Patent Application Publication
(10) Pub. No.: US 2023/0280321 A1
(43) Pub. Date: Sep. 7, 2023

(54) SYSTEM AND METHOD FOR REMOTE OZONE MONITORING WITHIN FRUIT HARVESTS

(71) Applicants: Grant Still Shatto, III, Castro Valley, CA (US); Christian DeBlasio, Belmon, CA (US)

(72) Inventors: Grant Still Shatto, III, Castro Valley, CA (US); Christian DeBlasio, Belmon, CA (US)

(21) Appl. No.: 17/653,648

(22) Filed: Mar. 5, 2022

(51) Int. Cl. G01N 33/00; G01N 33/02

(52) U.S. Cl. CPC G01N 33/0031; G01N 33/025; G01N 27/407

(57) ABSTRACT

A method and system are described for remotely measuring ozone and other atmospheric conditions within a post-harvest fruit mass.

### FIELD OF THE DISCLOSURE

The disclosure broadly relates to a system and method for remotely measuring atmospheric conditions, specifically ozone (O3) within a fruit harvest for the means of monitoring the fruit properties from within and controlling the atmosphere the fruit is exposed to by measuring the internal conditions of the fruit.

### BACKGROUND OF THE DISCLOSURE

Ozone has many uses in the post-harvest process of fruit. It is used to slow ripening, reduce human health risks by deactivating molds, bacteria, and viruses. It is also used to lower pesticides, fungicides, and sulfur dioxide applied to fruit before harvest, just to name a few benefits.

Temperature, humidity, and other atmospheric gas measurements in addition to ozone are equally important in tracking the condition of post-harvest fruit. Previous studies have focused on the atmosphere surrounding the post-harvest fruit, but this gives little control on the mass of the fruit since a majority of this fruit is not directly contacting the surrounding atmosphere. It is important to measure the atmospheric conditions within the interior of the fruit mass to monitor and control the fruit more accurately. This invention relates generally to a remote device used to measure ozone along with temperature, humidity, and atmospheric gases within the interior of a post-harvest mass of fruit held within a container.

### BRIEF DESCRIPTION OF THE DRAWINGS

FIG. 1 shows the results from a test performed on post-harvest wine grapes completed in October of 2021. The wine grapes were hand-picked into slotted containers each measuring 39.37″×47.24″×31.10″ (L×W×H, in inches). These bins were placed into a standard 40-foot refrigerated container for storage over a period of 20 hours. In the test, there were three ozone sensing system (OSS) sensors placed within one of the bins. Each sensor was calibrated identically and showed identical ozone concentration readings throughout the range tested.

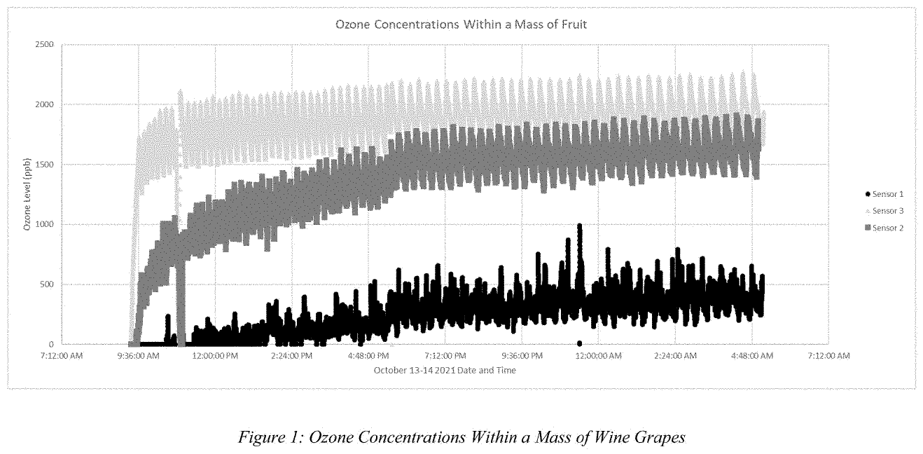

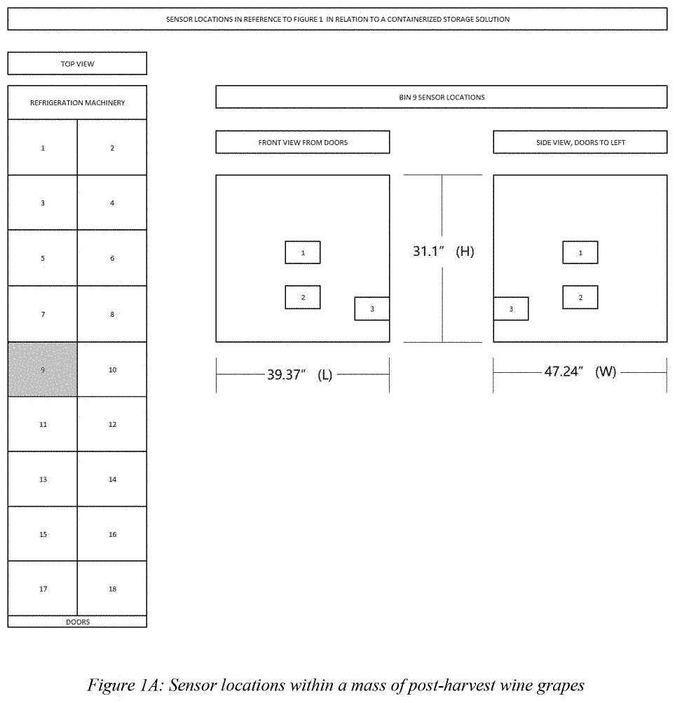

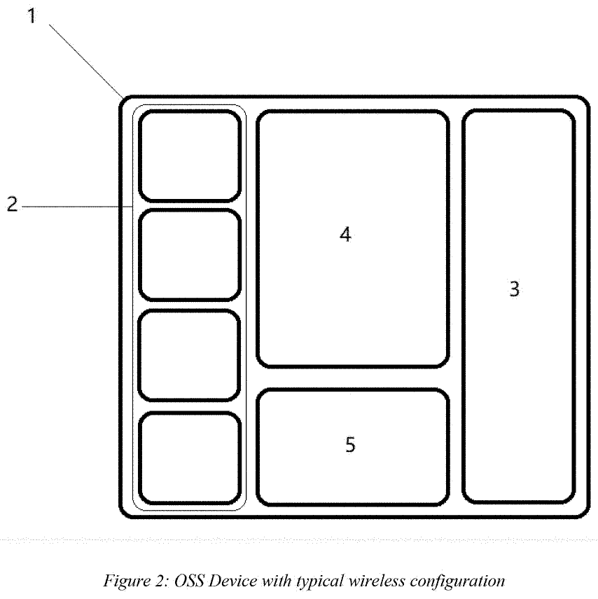

FIG. 1A shows the placement of the OSS sensors within the test that was performed that produced the data in FIG. 1. All three (3) of the OSS sensors were placed in bin nine (9) of the 40-foot refrigerated container. Bin 9 was chosen because it was at the center of the refrigeration chamber. The OSS sensor locations were chosen to show a difference in the ozone readings depending on their relative location to the exterior of the bin.
OSS sensor 1 was placed in the center of bin 9's footprint with the sensor 18 inches up from the bottom of the bin.
OSS sensor 2 was placed in the center of bin 9's footprint with the sensor 12 inches up from the bottom of the bin.
OSS sensor 3 was placed in the front right (if looking at the bin from the doors of the container) of bin 9, with it raised 10 inches off the bottom of the bin.

FIG. 2 shows the typical configuration of the OSS sensor device with its various components.
1. Component [1] shows the housing of the OSS made from a solid, waterproof material that protects the electronic components within from the atmospheric conditions the OSS may be subjected to.
2. Component [2] shows the sensing element(s) that are within the OSS housing, component [1]. The sensing element(s) are the instruments that measure the concentrations of the atmospheric conditions within the fruit mass that include ozone and any of the following additional conditions: temperature, humidity, oxygen, carbon dioxide, and ethylene.
3. Component [3] shows the power management unit that can include a battery, transforming circuitry, and a charging circuit. This component powers the rest of the components within the housing [1]. This component can also be bypassed or removed if the OSS is used as a wired unit.
4. Component [4] shows the controller of the OSS unit. The controller manages and takes the instrument reading(s) from component [2] and packages them up for transmitting via component [5]. The controller also manages component [2] and component [5]. Component [4] can also be bypassed or removed if the OSS is used as a wired unit
5. Component [5] is the remote transmitting equipment that is used to send the data that was initially collected by component(s) [2]. Component [5] can include many different types of wireless communication hardware that is further discussed in the following descriptions and claims. This component can also be bypassed or removed if the OSS is used as a wired unit.

### DETAILED DESCRIPTION

In this description and the following claims, “Ozone” (or “Trioxygen”) is defined as an inorganic molecule with the chemical formula O₃. The terms ozone and O₃ are used interchangeably throughout, this description, following claims, and figures and drawings. Ozone interferes with the metabolism of bacterium cells and can break through cell membranes that leads to the destruction of viruses, bacteria, mold cells and spores that aids in the preservation and shelf-life extension of post-harvest fruit.

In this description and the following claims, remote can also be described as a wired connection where the data collection center is tethered to the device through a cable or other physical means.

In this description and the following claims, data refers to a measurement taken from an instrument or sensing element within the device that can be read, monitored, or used to control a parameter surrounding the post-harvest fruit process. This can be in the form of a data packet transmitted wirelessly, serially printed over a wired connection, or an analog or digital output from the device itself.

In this description and the following claims, a process refers to a controlled system used to transport, house, or alter the post-harvest fruit mass. This can be in the form of a logistics process that transports the fruit, or a processing method that alters the shape or form or chemical makeup of the fruit to another form or product.

In this description and the following claims, a concentration refers to a measurement taken that can be in the form of a ratio, or percentage, either volumetrically, by mass; molecularly, or in reference to another known absolute value. For the purposes of this invention, ozone concentrations are measured between 0 and 20,000 parts per billion (ppb) or equivalent concentrations within the fruit mass.

In this description and the following claims, a fruit mass refers to any amount of fruit other than one (1) piece of fruit, or any homogeneous collection of more than once (1) piece of fruit.

In this description and the following claims, a measuring apparatus can comprise an ozone sensing element using electrochemical, ultraviolet, or infrared based sensing technology designed to measure ozone concentrations in a fluid media.

In this description and in the following claims, we reference ‘fruit’ as an individual item or an assembly of fruit items from a tree or a vineyard. We also reference ‘fruit’ as at least one of the following: wine grapes, table grapes, peaches, nectarines, plums, lychees, mangoes, almonds, pistachios, apricots, dates and cherries, strawberries, raspberries, blueberries, blackberries, red currants, white currants, blackcurrants, avocados, bananas, potatoes, onions, ginger, garlic, apples and pears.

In this description and the following claims, a post-harvest “container” can include open or closed type trucking containers that are both removable and fixed on trailer chassis. Fruit can also be picked by hand or by machine into harvest “bin” containers that are more easily transported within the fields and facilities where fruit is harvested and stored post-harvest.

A use for ozone was described by Bellincontro et al (Australian Journal of Grape and Wine Research, 2017) in their investigation using ozone to eliminate the use of sulfur dioxide added at the start of the wine-grape fermentation. The postharvest ozonation fumigation overnight of Petit Verdot grapes with 3 mg/liter (1400 ppm) eliminated the need for sulfur dioxide, significantly decreased the microbial count and increased the anthocyanin concentrations in the finished wines.

Ozone fumigation can have other effects beyond microbe elimination. Segade (Food Research International, 2019), for example, has shown changes in stilbenes, including trans-resveratrol and its derivatives, during ozone exposure of 60 ul/L (60 ppm) for 48 hours of “Moscato Bianco” wine grapes. In another study, Segade (Science Reports, 2017) observed an effect of ozone fumigation on the grape terpene profile at ozone exposure of 30 ul/L (30 ppm) over 24 days.

There are methods and devices currently on the market that do meet the demand of measuring some storage conditions within post-harvest fruit masses, but these methods and devices do not include measuring ozone concentration in the proximity of a fruit as a critical parameter.

Conventional types of direct fruit freshness sensors include spoilage, ripeness, leak, microbial pathogens, ethylene, and senescence indicators (Alam, Rathi, Beshai, Sarabha, & Deen, 2021). In most cases, the sensors contain a color indicator for easy visualization of freshness level by the naked eye. These indicators work off a chemical reaction to show when certain gasses in concentration were measured. Ethylene and other senescence indicators will turn color at lower concentrations because these will cause fruit spoilage at lower concentrations than oxygen, for example. Temperature and humidity of refrigerated fruit storage was also developed by two wireless sensing technologies, RFID and wireless sensor network. Real-time temperature, and humidity monitoring of a small cold storage of fruit and vegetable were demonstrated using an Arduino microcontroller-based temperature and humidity monitoring system (Tang, Tan, Chen, Li, & Shuai, 2020) The system hardware components included microcontroller unit, wireless communication protocols, temperature and humidity sensor, and organic light emitting diode (OLED) display. The system software components included Arduino IDE, UartAssist serial debugging assistant, and Lighting Blinker. The system test results showed that it can perform remote monitoring with high measurement accuracy and ease-of use.

Carbon dioxide detection and can be measured with an accuracy of +/−0.5% and a sensitivity of about 0.05% (FreshView, 2021), The same CO2 sensor can be reused in multiple fruit shipments, and minimal maintenance required. However, the optical detector is prone to external spectral interference, leading to inaccurate light detection, but this inaccuracy can be avoided by using optical filters (Dinh, Choi, Son, & Kim, 2016). A major drawback of this type of sensing (NDIR) sensing for smart packaging is that it is more suitable for system level smart packaging where the NDIR sensor is located outside the food package and provides feedback to system actuators to improve storage conditions.

No current study or currently available product exists that includes the measuring of ozone for use inside a container containing post-harvest fruit. All previous work has been done measuring the atmospheric conditions around the post-harvest fruit on the exterior of the container and not within. It is important to note that to achieve the results demonstrated by the prior art, it is necessary to ensure that the entirety of the post-harvest fruit has been exposed to the same homogeneous atmosphere required to achieve positive results.

The present invention, using a measuring apparatus referred to as the ozone sensing system (OSS), comprises a remote sensor whose primary function is to measure ozone concentrations within a mass of fruit directly after harvest. The device herein is designed to be placed within proximity of the fruit post-harvest without affecting fruit quality. The device is non-intrusive and designed to give further information to the user on the conditions that are within the fruit mass in order to further guide and improve the conditions and processing affecting shipment, post processing, and storage of the fruit.

Fruit can be placed in many types of different containments, “containers” after they are harvested. Depending on the process or the type of fruit, these different containers serve a purpose in many different aspects of the supply and logistics chain of fresh fruit. The present invention is designed in a way so that the fruit can be placed into these containers before or after the fruit is harvested or transferred into the containers. This is so that the user of this invention (the OSS) does not have to tune their logistics and post-harvest process to use this device. The containers holding the fruit mass may have a plurality of openings to allow gas exchange between, the interior and exterior of fruit mass. Said container may be stationary or mobile. If mobile, container may be moved either by truck or by special equipment designed to move such container.

We reference ‘exterior’ as a location that is outside of a mass of fruit. These containers can also be permanently attached, either to a facility or foundation, to serve as a larger storage container where the fruit is placed in large masses. The fixed or movable post-harvest containers are often refrigerated to aid in the post-harvest preservation of the fruit. This further extends the need for the current invention because the atmospheric conditions that are surrounding the fruit mass are controlled by equipment that is driven by instruments that are also located in the exterior of the fruit mass or in the refrigeration equipment itself.

An article from the Swiss Federal Laboratories and Materials Science and Technology (Zogg, 2017), teaches us that the atmospheric conditions within a fruit mass can vary widely between that of the exterior conditions

Although sensors measure the air temperature in the freight container, it is the core temperature of the individual fruit that is decisive for the quality of the fruit. However up to now, it has only been possible to measure this “invasively”, i.e. by inserting a sensor through the skin and into the center. And even this process has drawbacks. To carry out the measurement, the technician usually takes a piece of fruit from a cardboard box in the front row of pallets in the container, which in turn distorts the result for the entire fruit mass. Fruit that is closer to the outside of the transport container is better refrigerated than fruit on the inside. (Zogg, 2017)

Because ozone is typically distributed into the refrigeration or air movement system within a post-harvest container, the fruit that is closer to the outside of the container is exposed to higher, more uniform conditions than the fruit that is in the interior of the fruit, mass. This is illustrated in FIG. 1 which shows that the concentration of ozone within a mass of fruit held within a container differs greatly, depending on the location of the sensor within the container. All three sensors (OSS's) were calibrated identically and placed into the container prior to the fruit being placed into said container.

The current invention comprises an electronically powered instrument designed to be installed within the interior of a fruit mass (but not invasively) to allow the remote sensing of ozone and other atmospheric conditions. The form of the device is small enough to not displace a noticeable amount of volume since the container is primarily designed to hold post-harvest fruit and should remain its primary function. The typical configuration of the OSS and its components can be seen in FIG. 2.

The OSS device collects ozone measurements within the fruit mass using an ozone sensing element and this data can be stored locally or sent to an external system for the purpose of monitoring, controlling, or post processing of the data.

The OSS device comprises of a housing (Item [1] in FIG. 2) where within such housing the electronic portion of the device is mounted. The primary purpose of the housing is to protect the electronic portion of the device from the oxidizing atmosphere and external atmosphere that is surrounding it. The latter includes but is not limited to physical shock, water inclusion, crushing, and extreme temperatures. The housing [1] comprises of the housing itself and a sealing gasket that allows the device to be opened for service and calibration.

The electronic portion of the device incudes the sensing element(s) [2], power management electronics [3], a controlling unit or microprocessor “controller” [4], and the remote transmitting elements [5] needed to send the data to an external location where it can be useful to the user or equipment that is controlling the conditions around the post-harvest fruit.

The remote transmitting elements [5] of the OSS are capable of transmitting the data from the device to a remote location in a variety of different ways including radio frequency identification (RFID), IEEE 802.11b Direct Sequence (WIFI), General Packet Radio Service (GPRS) over 2G, 3G, 4G, and 5G bandwidths, IEEE 802.11b Direct Sequence (Bluetooth), and Long Range Low Power, Wide Area Network (LoRaWAN)

In another embodiment of this invention, the data “data” that is collected by the device within the fruit mass might include any of the following in addition to ozone (O₃): Carbon Dioxide (CO₂) concentration, Temperature, Humidity, ethylene (C₂H₄) concentration, oxygen (O₂) concentration, volatile organic compounds (VOCs), levels of particulate matter of different size forms (“PM_{2.5}”, “PM₅”, “PM₁₀”)

In another embodiment, of this invention, the housing and the electronic portion of the device are a single piece wherein the electronic portion and housing are integrated through molding or other process. This would eliminate the need for a gasket and allows the device to be one single piece to achieve long term operation.

This device can also be constructed in a way where a controller is not needed within the device if the device is capable of sending raw data to an external location that acts as a controller or processor.

### BIBLIOGRAPHY

Antolini, A., Forniti, R., Modesti, M., Bellincontrol, A., Catelli, C., & Mencarelli, F. (2020). First Application of Ozone Postharvest Fumigation to Remove Smoke Taint from Grapes. Ozone Science & Engineering, 1-9.
Bellincontro, A., Catelli, C., Cotarella, R., & Mencarelli, F. (2017). Postharvest ozone fumigation of Petit Verdot grapes to prevent the use of sulfites and to increase anthocyanin in wine. Australian Journal of Grape and Wine Research, 200-2006.
Chen, Z., Sun, Y., Qi, Y., Liu, L., & Zho, Y. (2019). Mechanistic and Kinetic Investigations on the Ozonolysis of Biomass Burning Products: Guaiacol, Syringol, and Cresol. International Journal of Molecular Sciences, 1-14.
Segade, S. R., Vincenzi, S., Giacosa, S., & Rolle, L. (2019). Changes in stilbene compostion during postharvest ozone treatment of “Moscato Bianco” winegrapes. Food Research International, 252-257.
Alam, A., Radii, P., Beshai, H., Sarabha, G., & Deen, M. (2021). Fruit Quality Monitoring with Smart Packaging. Sensors, 30. Retrieved from https://doi.org/10.3390/s21041509
Dinh, T., Choi, I., Son, Y., & Kim, J. (2016). A review on non-dispersive infrared gas sensors: Improvement of sensor detection limit and interference correction. Sensors.
FreshView. (2021). Retrieved from https://www.freshview.com.au/page/sensor_ec_co2
Segade, S., Vilanova, M., Giacosa, S. et al. (2017). Ozone Improves the Aromatic Fingerprint of White Grapes. Sci Rep 7, 16301. (n.d.).
Tang. X., Tan. C., Chen, A., Li. Z., & Shuai, R. (2020). Design and implementation of temperature and humidity monitoring system for small cold storage of fruit and vegetable based on Arduino. 2020, 1601. J. Phys. Conf. Ser.
Zogg, C. (2017, March 22). “Camouflage” sensor monitors fruit cargo. Retrieved from Empa: https://www.empa.ch/web/s604/fruit-sensor?inheritRedirect=true

1. A method for monitoring fruit properties of a fruit harvest comprising:
a) a fruit mass located within a specific volume or container;
b) a measuring apparatus for the remote measurement of ozone and other atmospheric conditions within the confines of said volume or fruit container;
c) wherein said measuring apparatus is located within the interior of said fruit mass while monitoring ozone concentration and other atmospheric conditions of said fruit harvest to ensure uniform treatment of said post-harvest fruit mass.

2. A method according to claim 1, wherein the fruit mass is comprised at least one of: wine grapes, table grapes, peaches, nectarines, plums, lychees, mangoes, almonds, pistachios, apricots, dates and cherries, strawberries, raspberries, blueberries, blackberries, red currants, white currants, blackcurrants, avocados, bananas, potatoes, onions, ginger, garlic, apples and pears.

3. A method according to claim 1, wherein the post-harvest fruit mass is comprised of more than one piece of fruit.

4. A method according to claim 1, wherein the post-harvest fruit mass is held in a container.

5. A method according to claim 1, wherein the container has a plurality of openings to allow gas exchange between the interior and exterior of fruit mass.

6. A method according to claim 1, wherein the measuring apparatus measure ozone concentrations between 0 and 20,000 parts per billion (ppb)

7. A method according to claim 1, wherein the measuring apparatus measures ozone concentration and humidity.

8. A method according to claim 1, wherein the measuring apparatus measures ozone concentration and temperature.

9. A method according to claim 1, wherein the measuring apparatus measures ozone concentration and carbon dioxide concentration.

10. A method according to claim 1, wherein the measuring apparatus measures ozone concentration and ethylene concentration.

11. A method according to claim 1, wherein the measuring apparatus measures ozone concentration and oxygen concentration.

12. A method according to claim 1, wherein the measuring apparatus measures ozone concentration and particle concentration.

13. A method according to claim 1, wherein the measuring apparatus measures ozone concentration and at least one of: humidity, temperature, carbon dioxide concentration, ethylene concentration, oxygen concentration, particle concentration.

14. A method according to claim 1, wherein the container is mobile in nature, either by truck or equipment designed to move such container.

15. A method according to claim 1, wherein the container is stationary in nature, such as a storage facility designed to hold and store post-harvest fruit.

16. A method according to claim 1, wherein the measuring apparatus is remote and can be wired to a controller.

17. A method according to claim 1, wherein the measuring apparatus is remote and wireless.

18. A method according to claim 1, wherein the measuring apparatus is powered internally to be truly wireless.

19. A method according to claim 11, wherein the measuring apparatus is powered externally.
